(19) United States
(12) Patent Application Publication
(10) Pub. No.: US 2009/0158472 A1
(43) Pub. Date: Jun. 18, 2009

(54) TRANSFORMANT PLANT

(75) Inventors: Daisuke SHIBATA, Kisarazu-shi (JP); Yoichi Ogawa, Kisarazu-shi (JP); Migiwa Takeda, Kisarazu-shi (JP); Tomonori Takashi, Kisarazu-shi (JP); Asuka Nishimura, Tokyo (JP)

Correspondence Address: ARENT FOX LLP, 1050 CONNECTICUT AVENUE, N.W., SUITE 400, WASHINGTON, DC, 20036, US

(73) Assignees: Kazusa DNA Research Institute, Kisarazu-shi (JP); HONDA MOTOR CO., LTD., Tokyo (JP)

(21) Appl. No.: 12/203,772

(22) Filed: Sep. 3, 2008

(30) Foreign Application Priority Data
Sep. 4, 2007 (JP) 2007-229270
Mar. 5, 2008 (JP) 2008-055493

(51) Int. Cl. A01H 5/00

(52) U.S. Cl. 800/306; 800/298; 800/320.2

(57) ABSTRACT

A transformant plant transformed with an expression vector, the expression vector including a nucleotide sequence encoding a first polypeptide which has thermophilic endo-1,4-beta-glucanase activity, so that the polypeptide is capable of being expressed in a host cell of the transformant plant.

Priority is claimed on Japanese Patent Application No. 2007-229270, filed Sep. 4, 2007, and Japanese Patent Application No. 2008-055493, filed Mar. 5, 2008, the contents of which are incorporated herein by reference.

### BACKGROUND OF THE INVENTION

1. Field of the Invention

The present invention relates to transformant plants.

2. Description of Related Art

Recently, methods of biomass ethanol (bioethanol) production are intensively studied in many countries. Biomass ethanol is produced from plant resources by saccharizing them into monosaccharides by enzyme treatments or the like, and then the material is subjected to alcoholic fermentation by microorganisms such as yeast. Biomass ethanol is highly expected as a major energy source in the future, in view of the growing concern about the global warming, since biomass ethanol is a natural energy produced from regenerative plant resources, and it does not increase carbon amount in the carbon cycle on the global surface, when it is burned.

For biomass ethanol production, plant resources such as sugar cane, corn, and the like, which contain rich sugar or starch, have been used, in order to achieve superior production efficiency. However, since those kind of plant resources are also used as food, it is desired to develop production method of biomass ethanol using plant resources which are not directly used as food. For example, it is expected that by using non-edible plants such as weeds, and agricultural wastes including non-edible portions of edible plants, such as rice straw, it becomes possible to consistently produce biomass ethanol at a lower cost.

A major component of plant tissue, cellulose, is a chain polymer consisting of a plurality of β-1 to 4 linked D-glucose units. That is, if cellulose can be utilized efficiently as raw material to produce biomass ethanol, it becomes possible to utilize cellulose-rich biomass as a raw material of biomass ethanol production at a high-yield, comparable to the yield when sugar cane or the like is used.

One of main causes of the non-ideal yield of the biomass ethanol production using a raw material of cellulose-rich biomass, is the difficulty of saccharization of cellulose-rich biomass when compared to that of starch-rich plant materials. Accordingly, by improving the saccharization efficiency of the cellulose-rich biomass, improved production yield of the biomass ethanol can be expected. Usually, the saccharization of the cellulose-rich biomass is performed by a hydrolysis using enzymes, acid or alkaline solutions, or pressured hot water. Particularly, by using enzymes such as cellulase, it is possible to perform saccharization at a gentle reaction condition.

Cellulase is a general term for enzymes which break down cellulose into cellobiose or glucose units. Cellulases are categorized, by their catalyzation methods, into endoglucanases, exoglucanases, and β-glucosidases. Particularly, endoglucanases (endo-1,4-beta-glucanase; EC 3.2.1.4) is a group of enzymes which hydrolyze glycoside bonds of 1,4-beta-glucans such as celluloses, and are particularly important in the cellulose hydrolyzation. In general, efficiencies of chemical reactions such as hydrolyzation becomes higher at a higher temperature. Accordingly, by using endoglucanases derived from hyperthermophilic bacterium such as bacterium of genus Pyrococcus, an improvement can be expected in the yield of saccharization procedure of cellulose-rich biomass (for example, refer to Japanese Unexamined Patent Application, First Publication No. 2003-210182, Japanese Unexamined Patent Application, First Publication No. 2004-105130, and Japanese Unexamined Patent Application, First Publication No. 2005-27572).

However, since enzymes such as endoglucanase are generally expensive, it is not economically desirable to use large amounts of such enzymes for saccharization procedures of cellulose-rich biomass.

Moreover, raw cellulose-rich biomass is not suitable substrate of enzyme catalyzation procedures. Therefore, in order to efficiently perform enzyme reactions, pretreatments, such as physical treatments including milling and steaming, or chemical treatments by acids and alkaline, are necessary. The costs of those pretreatments have been problems.

An object of the present invention is to provide plants which have high expression amount of thermophilic endo-1,4-beta-glucanase.

As a result of intensive investigation in order to achieve the above object, the inventors of the present invention found that transformant plants expressing polypeptides having thermophilic endo-1,4-beta-glucanase activity has a high expression amount of thermophilic endo-1,4-beta-glucanase. The inventors found that such transformant plants can simultaneously produce both cellulose, as raw material of biomass ethanol, and thermophilic endo-1,4-beta-glucanase suitable for hydrolyzation of the cellulose.

### SUMMARY OF THE INVENTION

In order to achieve the above object, the present invention employed the following.

(1) A transformant plant transformed with an expression vector, the expression vector including a nucleotide sequence encoding a first polypeptide which has thermophilic endo-1,4-beta-glucanase activity, so that the polypeptide is capable of being expressed in a host cell of the transformant plant.

(2) It may be arranged such that, in the transformant plant: the first polypeptide further includes an amino acid sequence of a chitin binding domain of a chitinase.

(3) It may be arranged such that, in the transformant plant: the first polypeptide is a polypeptide having an amino acid sequence selected from the group consisting of: (a) an amino acid sequence set forth in SEQ ID NO: 2; (b) an amino acid sequence set forth in SEQ ID NO: 2 including substitution, deletion, insertion, and/or addition of one or several of amino acids in the amino acid sequence.

(4) It may be arranged such that, in the transformant plant: the first polypeptide is a polypeptide having an amino acid sequence selected from the group consisting of: (a) an amino acid sequence set forth in SEQ ID NO: 4; (b) an amino acid sequence set forth in SEQ ID NO: 4 including substitution, deletion, insertion, and/or addition of one or several of amino acids in the amino acid sequence.

(5) It may be arranged such that, in the transformant plant: the first polypeptide is a polypeptide having an amino acid sequence selected from the group consisting of: (a) an amino acid sequence set forth in SEQ ID NO: 6; (b) an amino acid sequence set forth in SEQ ID NO: 6 including substitution, deletion, insertion, and/or addition of one or several of amino acids in the amino acid sequence.

(6) It may be arranged such that, in the transformant plant: the nucleotide sequence is selected from the group consisting of: (a) a nucleotide sequence set forth in SEQ ID NO: 7; (b) a nucleotide sequence set forth in SEQ ID NO: 7 including substitution, deletion, insertion, and/or addition of one or several of nucleotide in the nucleotide sequence.

(7) It may be arranged such that, in the transformant plant: the first polypeptide is a polypeptide having an amino acid sequence selected from the group consisting of: (a) an amino acid sequence set forth in SEQ ID NO: 10; (b) an amino acid sequence set forth in SEQ ID NO: 10 including substitution, deletion, insertion, and/or addition of one or several of amino acids in the amino acid sequence.

(8) It may be arranged such that, in the transformant plant: the first polypeptide further includes an apoplastic-transfer signal peptide at the amino-tenninus thereof.

(9) It may be arranged such that, in the transformant plant: the first polypeptide further includes an endoplasmic reticulum localization signal peptide at the carboxyl-terminus thereof.

(10) It may be arranged such that, in the transformant plant: the plant belongs to family Brassicaceae.

(11) It may be arranged such that, in the transformant plant: the plant is Arabidopsis thaliana.

(12) It may be arranged such that, in the transformant plant: the plant belongs to family Poaceae.

(13) It may be arranged such that, in the transformant plant: the plant is rice.

The transformant plant of the present invention expresses polypeptides having an activity of thermophilic endo-1,4-beta-glucanase (hereinafter, referred to as thermophilic endoglucanase). Accordingly, cellulose, which is the major component of the plant, can be readily hydrolyzed at a high temperature condition. Therefore, by using the transformant plant of the present invention as a plant resource for a raw material of the biomass ethanol, the enzyme amount required in a saccharization procedure can be reduced significantly. Moreover, a pretreatment before the saccharization procedure can also be simplified. That is, the transformant plant of the present invention can simultaneously provide both cellulose and thermophilic endoglucanase suitable for cellulose hydrolyzation, rendering itself as a plant material particularly suitable for a raw material of biomass ethanol.

### BRIEF DESCRIPTION OF THE DRAWINGS

FIG. 1A is a conceptual diagram of an expression vector to obtain a transformant plant of the present invention, according to a first embodiment of the present invention.

FIG. 1B is a conceptual diagram of an expression vector to obtain a transformant plant of the present invention, according to a first embodiment of the present invention.

FIG. 1C is a conceptual diagram of an expression vector to obtain a transformant plant of the present invention, according to a first embodiment of the present invention.

FIG. 2 is a graph showing thermophilic endoglucanase activities of crude enzyme extracts of transformant Arabidopsis thaliana (hereinafter, referred to as Arabidopsis) obtained using apoplast-accumulation-type constructs of the second embodiment.

FIG. 3 is a graph showing thermophilic endoglucanase activities of crude enzyme extracts of transformant Arabidopsis obtained using apoplast-accumulation-type constructs with endoplasmic reticulum localization signal of the second embodiment.

FIG. 4 is a graph showing thermophilic endoglucanase activities of crude enzyme extracts of transformant Arabidopsis obtained using apoplast-accumulation-type constructs, and apoplast-accumulation-type constructs with endoplasmic reticulum localization signal of the fifth embodiment.

### DETAILED DESCRIPTION OF THE INVENTION

The transformant plant of the present invention is transformed using an expression vector which includes nucleic acid encoding polypeptide having thermophilic endoglucanase activity. The expression vector can express a polypeptide having thermophilic endoglucanase activity in the host cells.

According to the transformant plant of the present invention, since polypeptides with thermophilic endoglucanase activity are expressed, by performing, for example, a heating treatment at a temperature on or above 85° C., the cellulose in the transformant plant can be discomposed. At a normal plant cultivation temperatures at or below 50° C., the polypeptides have only limited endoglucanase activities. Accordingly, the transformant plant of the present invention can be cultivated normally as non-transformant plants of the same species.

For the polypeptides having thermophilic endoglucanase activity of the present invention, any polypeptide having an enzyme activity to hydrolyze glycoside bonds of 1, 4-beta-glucans including cellulose, at a temperature on or above 85° C. may be used.

One example of such polypeptides may be endoglucanases or the like derived from thermophilic microorganisms which survive in a high-temperature environment.

Examples of such thermophilic microorganisms include, thermophilic bacterium including Pyrococcus, Aeropyrum, Sulfolobus, Thermoplasma, Thermoproteus, Bacillus, Synechococcus, and Thermus.

Moreover, the polypeptides having thermophilic endoglucanase activity of the present invention may be a polypeptides having a thermophilic endoglucanase activity and having an amino acid sequence constituting thermophilic endoglucanase, in which one or more of amino acid residues are deleted, replaced, or added to/from the sequence.

As long as the polypeptide maintains the thermophilic endoglucanase activity, the positions or kinds of the amino acid residue modifications are not limited.

In the present invention, a DNA with nucleotide sequence encoding polypeptides having a thermophilic endoglucanase activity may be obtained, for example, by extracting nucleic acids from cultures of microorganisms having thermophilic endoglucanase, and then performing PCR (polymerase chain reaction) or hybridization procedure to the extracted nucleic acids, using primers and probes designed based on information of nucleotide sequence encoding thermophilic endoglucanase.

Moreover, as for the polypeptides having a thermophilic endoglucanase activity and having an amino acid sequence constituting thermophilic endoglucanase, in which one or more of amino acid residues are deleted, replaced, or added to/from the sequence, DNA encoding such polypeptides may be acquired by modifying DNA of nucleotide sequence encoding thermophilic endoglucanase, by known genetic recombination techniques.

The information of nucleotide sequence encoding thermophilic endoglucanase may be obtained from any of international nucleotide sequence databases, including GenBank, DDBJ, EMBL.

Moreover, by using known information of nucleotide sequence encoding thermophilic endoglucanase, such as the nucleotide sequence of SEQ ID NO: 1, and performing known methods such as BLAST search or the like, nucleotide sequence information which is likely to encode polypeptides having thermophilic endoglucanase activity can be obtained. SEQ ID NO: 1 is the nucleotide sequence encoding wild type thermophilic endoglucanase derived from Pyrococcus horikoshii.

To determine whether or not DNA fragments obtained by such methods actually encodes polypeptides having thermophilic endoglucanase activity, expression vectors including such DNA fragments may be constructed. The expression vectors may be introduced into appropriate host cells such as Escherichia coli, to express the content of DNA fragments. The thermophilic endoglucanase activity of the resulting polypeptides may be measured using known methods such as Somogyi-Nelson method.

As for the polypeptides having thermophilic endoglucanase activity of the present invention, it is preferable to use thermophilic endoglucanase derived from microorganisms of genus Pyrococcus. It is further preferable to use one derived from Pyrococcus horikoshii. It is further preferable to use any one of polypeptides of SEQ ID NO: 2, 4, 6. Polypeptides of SEQ ID NO: 2, 4, 6 may include deletion, substitution, or addition to/from any position in those sequences by one or more amino acid residues, while retaining the thermophilic endoglucanase activity.

The polypeptide having amino acid sequence of SEQ ID NO: 2 is wild type thermophilic endoglucanase derived from Pyrococcus horikoshii. The thermophilic endoglucanase has the optimum temperature for the enzyme activity of 97° C., the optimum pH of 5.4 to 6.0. The endoglucanase is stable after heating at 97° C. for three hours.

Furthermore, the polypeptide having amino acid sequence of SEQ ID NO: 4 (hereinafter, referred to as EGPh) is a modified polypeptide from SEQ ID NO: 2, with a deletion of a signal peptide (position 2 to 28 of the amino acid sequence of SEQ ID NO: 2).

Moreover, the polypeptide having amino acid sequence of SEQ ID NO: 6 (hereinafter, referred to as EGPf) is a modified polypeptide from SEQ ID NO: 4, with a deletion of 42 amino acids at the carboxyl terminus. The term ‘carboxyl terminus’ will be referred to as ‘c-terminus’, hereinafter. By deleting the c-terminus amino acids of SEQ ID NO: 4, an expression amount of the thermophilic endoglucanase in the transformant plant can be increased (see, for example, Japanese Unexamined Patent Application, First Publication No. 2004-105130).

The nucleotide sequence encoding EGPh and EGPf may be a homologous sequence to the corresponding position of SEQ ID NO: 1. It may also be a modified nucleotide sequence of SEQ ID NO: 1, with one or more of the nucleic acids deleted, modified, or inserted, while the encoded polypeptide retaining the thermophilic endo-1,4-beta-glucanaseactivity. For example, it is preferable that the nucleotide sequence encoding the EGPf is that of SEQ ID NO: 7, which is the nucleotide sequence of SEQ ID NO: 1 with a substation at positions 822 to 825 from agga to tggt. This way, the expression amount of the EGPf in the transformant plant can be increased without changing the amino acid sequence of EGPf. The nucleotide sequence agga is the second SD-like sequence counted from the n-terminus. By modifying the SD-like sequence, the translation efficiency is expected to increase.

The polypeptides having a thermophilic endoglucanase activity of the present invention may be a polypeptide constituted by peptide-linking a polypeptide having a thermophilic endoglucanase activity (hereinafter, called a first peptide) and a polypeptide having a function other than thermophilic endoglucanase activity (hereinafter, called a second peptide). The second peptide is not limited to a particular peptide unless it inhibits the thermophilic endoglucanase activity of the first peptide. The second peptide is not necessarily thermophilic. It is possible to have either of the first peptide or the second peptide at the n-terminus of the linked peptide, although it is preferable that the first peptide is at the n-terminus. It is also possible to have a spacer peptide between the first and second peptides.

It is preferable that the second peptide is a polypeptide having an amino acid sequence of a chitin binding domain of a chitinase. By adding the chitin binding domain to the thermophilic endoglucanase, it is possible to improve the enzyme activity of the thermophilic endoglucanase. The chitinase may be thermophilic or thermostable, although it is preferable that the chitinase is thermophilic. A chitin binding domain of a chitinase generally has 50 to 150 amino acid residues. It is preferable that a chitin binding domain to be added to a thermophilic endoglucanase posses the whole region of the chitin binding domain. It is possible that the chitin binding domain has a partial deletion. Moreover, the chitin binding domain can be consisting of a plurality of chitin binding domain sequences derived from same or different species. It is preferable that the second peptide is derived from Pyrococcus furiosus. SEQ ID NO: 8 is the amino acid sequence of chitinase derived from Pyrococcus furiosus. The chitin binding domain thereof is considered to be reside in amino acid sequence regions of positions 70 to 140 and positions 600 to 720.

For a polypeptide having thermophilic endoglucanase activity of the present invention, connected with a polypeptide having amino acid sequence of chitin binding domain of chitinase, it is preferable to use the polypeptide of SEQ ID NO: 10, or a polypeptide with one or more amino acid residue deleted, substituted, or added thereto. SEQ ID NO: 10 is a EGPf with a chitin binding domain of a chitinase derived from Pyrococcus furiosus added at the c-terminus thereof (referred to as EGPfChiCBM, hereinafter). In the amino acid sequence of SEQ ID NO: 10: positions 1 to 389 are the amino acid sequence of SEQ ID NO: 6; positions 390 to 392 are the amino acid sequence of spacer peptide; positions 393 to 500 are the amino acid sequence of positions 613 to 720 of SEQ ID NO: 8.

The polypeptides having thermophilic endoglucanase activity of the present invention may further include a signal peptide which can localize the expressed polypeptides to a particular region of the plant cell. Examples of such signal peptides include, apoplastic-transfer signal peptide, endoplasmic reticulum localization signal peptide, nuclear transfer signal peptide, and secretion signal peptide. It is preferable that the polypeptides having thermophilic endoglucanase activity of the present invention possesses an apoplastic-transfer signal peptide at the n-terminus, or an endoplasmic reticulum localization signal peptide at the c-terminus, or both of them. By adding such signal peptides, the thermophilic endoglucanase activity of the polypeptides expressed in the transformant plant can be increased. It is assumed that the reasoning of such phenomenon is because, by such addition, the polypeptides are protected from digestion by cellular proteases. Moreover, it is also expected that, by adding the apoplastic-transfer signal peptide, the polypeptides having the thermophilic endoglucanase activity can be localized in the vicinity of the cell wall, which contains a large amount of cellulose. Therefore, the saccharization procedure can be performed efficiently, when the transformant plant of the present invention is used as the raw material of the biomass ethanol production.

The apoplastic-transfer signal peptide is not limited to a particular peptide sequence, as long as it is an apoplast transfer signal, and any known apoplastic-transfer signal peptide may be used. One example of such apoplastic-transfer signal peptide is, the signal peptide of the protease inhibitor II derived from potato, having an amino acid sequence of MDVHKEVNFVAYLLIVLGLLVLVSAMEHVDAKAC (see, for example, Wang, M., Goldstein, C., Su, W., Moore, P. H., Albert, H. H. 2005. Production of biologically active gm-csf in sugarcane: a secure biofactory. Transgenic Research. V14:P167-178.).

The endoplasmic reticulum localization signal peptide is not limited to a particular peptide sequence, as long as it can localize the polypeptide in the endoplasmic reticulum. Accordingly, any known endoplasmic reticulum localization signal peptide may be used. An example of such endoplasmic reticulum localization signal peptide is a signal peptide including the amino acid sequence of HDEL.

In the present invention, the expression vector includes nucleotide sequence encoding polypeptides with thermophilic endoglucanase activity. The expression vector can express a polypeptide having thermophilic endoglucanase activity in the host cell. That is, in the expression vector, a nucleotide sequence encoding such polypeptide is incorporated in the way the polypeptide can be expressed. Specifically, it is necessary that the expression vector is provided with an expression cassette including, from the upstream of the sequence, a promoter sequence, a sequence encoding a polypeptide having thermophilic endoglucanase activity, and a terminator sequence. Such parts of DNA sequences can be incorporated into the expression vector using known recombinant DNA procedures.

The expression vector in the present invention is not limited to any particular expression vector, as long as it includes a promoter sequence by which a transcription can be performed in plant cells, and a terminator sequence having a polyadenylation signal. Any common expression vector used for transformant plant cells or transformant plant may be adopted. Examples of such expression vectors include binary vectors such as pIG121 and pIG121Hm. Examples of such promoters include a nopalin synthetase gene promoter, and a cauliflower mosaic virus 35S RNA gene promoter. An example of terminators which can be used in the present invention is a nopalin synthetase gene terminator. Promoters specific to any tissue or organ may also be used. By using such tissue specific promoters, polypeptide having thermophilic endoglucanase activity can be expressed, not in the whole plant body, but in a specific tissue or an organ. Accordingly, it is assumed to be possible to express the polypeptide, for example, only in the non-edible portions of edible plants.

It is preferable that the expression vector includes not only the nucleotide sequence of the polypeptide having thermophilic endoglucanase activity, but also drug resistance genes and the like. In this case, the transformant plants having the expression vector can be readily selected out of non-transformant plants. Examples of the drag resistance genes include kanamycin resistance gene, hygromycin resistance gene, and bialaphos resistance gene.

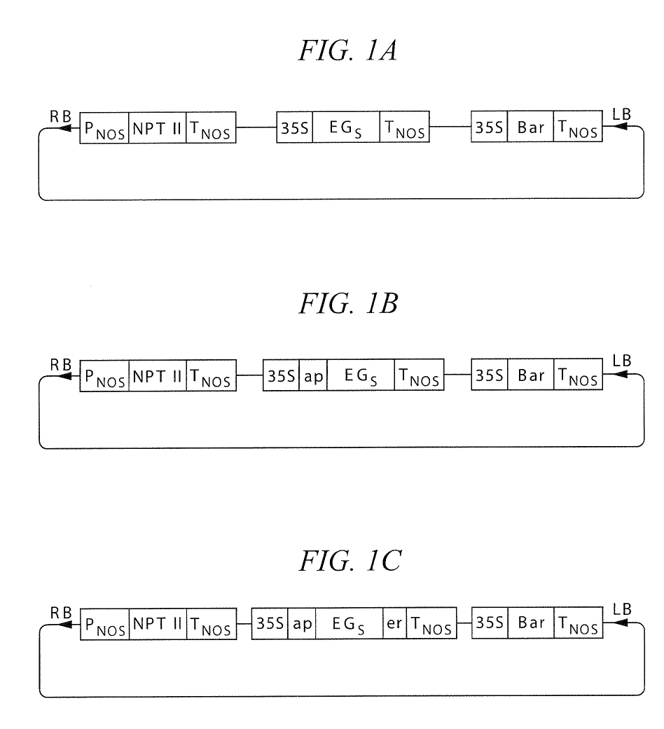

For example, by performing transformation procedure using one of expression vectors as shown in FIG. 1A to 1C, transformant plants of the present invention can be acquired. FIG. 1A is a schematic diagram of one of vectors constructed using a vector pIG121 Bar, in which a bialaphos resistance gene (Bar) is incorporated in the hygromycin resistance gene region of the binary vector pIG121Hm. In the construct of FIG. 1A, a nucleotide sequence encoding a polypeptides having thermophilic endoglucanase activity (EGs) is incorporated into an intron GUS region of pIG121Bar.

The construct is an expression vector having, from the upstream thereof, a kanamycin resistance gene expression cassette, an expression cassette for a polypeptide having thermophilic endoglucanase activity, and a bialaphos resistance gene expression cassette.

Each of FIGS. 1A to 1C shows an aspect of expression vector by which transformant plant of the present invention is obtained.

In the figures: P_{NOS} represents a nopalin synthetase gene promoter; NPT II represents a kanamycin resistance gene; T_{NOS} represents a nopalin synthetase gene terminator; 35S represents a cauliflower mosaic virus 35S RNA gene promoter; EGs represents a nucleotide sequence encoding a polypeptide having thermophilic endoglucanase activity; Bar represents a bialaphos resistance gene; ap represents a nucleotide sequence encoding an apoplastic-transfer signal peptide; er represents a nucleotide sequence encoding an endoplasmic reticulum localization signal peptide, respectively.

The kanamycin resistance gene expression cassette includes: a nopalin synthetase gene promoter (P_{NOS}); a kanamycin resistance gene (NPT II) connected to the downstream thereof; and a nopalin synthetase gene terminator (T_{NOS}) connected to the downstream thereof. The expression cassette of the polypeptide having thermophilic endoglucanase activity includes: a cauliflower mosaic virus 35S RNA gene promoter (35S); a nucleotide sequence encoding a polypeptide having thermophilic endoglucanase activity (EGs) connected to the downstream thereof; and a nopalin synthetase gene terminator connected thereto. The expression cassette of the bialaphos resistance gene includes: a cauliflower mosaic virus 35S RNA gene promoter; a bialaphos resistance gene (Bar) connected to the downstream thereof; and a nopalin synthetase gene terminator connected thereto.

FIG. 1B shows a variation of the vector shown in FIG. 1A, constructed by inserting a nucleotide sequence encoding apoplastic-transfer signal peptide (ap), at the 5′ terminus of the nucleotide sequence encoding the polypeptide having thermophilic endoglucanase activity.

FIG. 1C shows a variation of the vector shown in FIG. 1B, constructed by inserting a nucleotide sequence encoding endoplasmic reticulum localization signal peptide (er), at the 3′ terminus of the nucleotide sequence encoding a polypeptide having thermophilic endoglucanase activity.

In the present invention, the method of obtaining a transformant plants using the expression vectors is not limited to any particular method. It can be performed using any methods commonly used to prepare transformant plant cells and transformant plants. It is preferable to use, for example, agrobacterium method, particle-gun method, electroporation method, or PEG (polyethylene glycol) method. Among those methods, agrobacterium method is particularly preferable. The transformant plant cell and transformant plants can be selected using a drug resistance as a criteria. Cultured plant cells may be used as the host, as well as plant organs and plant tissues.

By using known plant tissue culture methods, it is possible to obtain transformant plants from the transformant plant cells, callus, and the like. For example, the transformant plant cells can be cultivated in hormone-free regeneration medium. The resulting young plant with root can be transplanted onto soil or the like and further cultivated, to obtain transformant plant.

Furthermore, the transformant plant of the present invention includes, in addition to the plants obtained directly by transformation, progeny plants thereof, which express the polypeptide having thermophilic endoglucanase activity as well. The progeny plants include plants obtained by germinating seeds from the parent plants, and also plants obtained by cutting propagation.

The species of the transformant plant of the present invention is not limited to any particular species. The species may belong to angiosperm, gymnosperm, Pteridophyta, or Bryophyte. Examples of the transformant plant of the present invention include plants belonging to, Brassicaceae, Poaceae, Solanaceae, Fabaceae, Asteraceae, Convolvulaceae, Euphorbiaceae, and the like. It is preferable to use plants of Brassicaceae and Poaceae, since they are suitable for transformation procedures using agrobacterium. Examples of plants of Brassicaceae include, thale cress (Arabidopsis thaliana), rapeseed, shepherd's-purse, daikon, cabbage, wasabi and the like. Examples of plants of Poaceae include, rice, corn, sorghum, wheat, barley, rye, Japanese barnyard millet and the like. Examples of plants of Solanaceae include, eggplant, potato, tomato, green pepper, tobacco, and the like. Examples of plants of Fabaceae include, peanut, chick-pea, soybean, common bean, and the like. Examples of plants of Compositae include, burdock, mugwort, pot marigold, cornflower, sunflower, and the like. Examples of plants of Convolvulaceae include, false bindweed (Calystegia japonica), Calystegia soldanella, dodder, field bindweed, and the like. Examples of plants of Euphorbiaceous include, spurge, Euphorbia sieboldiana, Euphorbia pekinensis Rupr, and the like.

It is preferable to use Arabidopsis for the transformant plant of the present invention. This is because Arabidopsis is one of so-called weeds, and is easy to cultivate. Arabidopsis also is an annual plant and has a short life cycle. In order to obtain Arabidopsis plant as a transformant plant of the present invention, for example, solution of agrobacterium transformed with an expression vector which can express polypeptide having thermophilic endoglucanase activity is prepared, and applied to a bud of the Arabidopsis plant body. After the infection of the agrobacterium, by using a floral dip method, in which transformant seeds are selected using antibiotics or the like, Arabidopsis plants as transformant plants of the present invention can be obtained.

It is also preferable to use rice for the transformant plant of the present invention. Rice is one of major agricultural product, and a large amount of inedible parts of rice, such as straw is wasted yearly as agricultural waste. By adopting the transformant rice according to the present invention as food crops, produced rice straws and the like can be converted to raw material of biomass ethanol production. Accordingly, it is possible to reduce the amount of agricultural waste significantly.

Transformant rice according to the present invention can be obtained by transforming an expression vector which can express a polypeptide having thermophilic endoglucanase activity. The transformation can be performed using the method of Nishimura et al. (See Nishimura et al. 2006, Nature Protocols 1, 2796-2802). Specifically, for example, a callus may be prepared by incubating a mature seed after removing the outer shell and sterilizing the surface thereof. The callus is then soaked in a solution of agrobacterium transformed by an expression vector which can express a polypeptide having thermophilic endoglucanase activity. Then transformed calluses are selected using antibiotics and the like, to obtain transformant rice plants according to the present invention.

It is also possible to obtain polypeptide having thermophilic endoglucanase activity, by extraction from the transformant plant of the present invention. Methods for the extraction is not limited to any particular extraction method, as long as it does not compromise the thermophilic endoglucanase activity of the polypeptide. The extraction can be performed using any methods commonly used to extract polypeptides from cells or biological tissues. Examples of such extraction methods include, the method of Kawazu et al. (see Kawazu et al., 1999, Journal of Bioscience and Bioengineering, 88, pp. 421-425), and the method of Kimura et al. (Kimura et al. 2003, Applied microbiology and biotechnology. 62, 374-379).

The transformant plant of the present invention is particularly suitable as raw material for biomass ethanol production. Such biomass ethanol production can be performed, for example, by the following procedure. First, the transformant plant of the present invention is subjected to a pretreatment, and a suitable buffer is added thereto, and the mixture is heated at a temperature at or above 85° C. By such heating treatment, the polypeptide having thermophilic endoglucanase activity, which is expressed in the transformant plant, functions efficiently, and digests the cellulose in the transformant plant, yielding a saccharized extract. The saccharized extract is then inoculated with yeast or the like, to perform alcoholic fermentation, and thereby produce biomass ethanol.

It is preferable that the pretreatment is performed by a method which preserves the thermophilic endoglucanase activity of the polypeptide. For example, physical treatments including milling or heating are preferable. On the other hand, treatments with a strong acid or a strong alkaline may be avoided since those treatments could inactivate the polypeptide. The buffer is not limited to a particular kind, as far as it is suitable for the cellulose hydrolyzation reaction by the polypeptide. The buffer may contain detergents or other enzyme which can enhance the digestion of transformant plant. For example, when the polypeptide is the thermophilic endoglucanase derived from Pyrococcus horikoshii, it is preferable to use a buffer of pH 5 to 6. This is because the optimum pH of the thermophilic endoglucanase is in the range of pH 5 to 6.

Moreover, the biomass ethanol may be produced using the polypeptide having thermophilic endoglucanase activity extracted from the transformant plant of the present invention. For example, the transformant plant of the present invention may be subjected to a pretreatment, and soaked in an appropriate extraction buffer, to extract the polypeptide having thermophilic endoglucanase activity. Thereafter, the crude extract is separated into an extracted polypeptide and a residual plant material. The separated residual plant material is subjected to further pretreatment, and thereafter, mixed back with the extracted polypeptide. The mixture is then heated at a temperature on or above 85° C., and thereby the cellulose contained in the residual plant material is hydrolyzed, to obtain saccharized solution.

The extraction buffer used to extract the polypeptide is not limited to a particular buffer, as far as it can extract the polypeptide without inactivating the thermophilic endoglucanase. However, an extraction buffer including a solubilizing agent such as detergents and the like is preferable. In this way, the digestion of the transformant plant and the like is enhanced, and thereby the extraction efficiency of the polypeptide is improved. For example, when the polypeptide is the thermophilic endoglucanase derived from Pyrococcus horikoshii, it is preferable to use an extraction buffer of pH 5 to 6, which includes a detergent such as TritonX-100.

Moreover, the method of separating the extracted polypeptide and the residual plant material is not limited to a particular method. It may be any one of common methods used in separation procedures to extract a particular chemical component from biological solid material of plant or the like. Examples of such methods include, squeezing transformant plant soaked in an extraction buffer, filtering the material using a coarse filter, and centrifugation method. When the amount of the material is large, it is also preferable to perform a compression filtration.

The pretreatment of the residual plant material is not limited to a particular treatment, as far as it can facilitate the saccharization procedure. Any pretreatment commonly used for biomass material may be used. Examples of such pretreatments include, heating, chemical treatments such as acid or alkaline treatment. Specifically, alkaline treatment is preferable. When the polypeptide having thermophilic endoglucanase activity is extracted from the transformant plant in advance, a variety of pretreatments can be performed to the residual material, without a concern of losing the enzyme activity.

Although examples of embodiments of the present invention are shown below to further explain the present invention, the scope of the present invention is not limited to the embodiments.

### First Embodiment

### Preparation of Transformant Arabidopsis

A transformant Arabidopsis is obtained using an expression vector having a nucleotide sequence encoding a polypeptide with thermophilic endoglucanase activity.

For the expression vector, apoplast-accumulation-type constructs as shown in FIG. 1B, and apoplast-accumulation-type constructs with an endoplasmic reticulum localization signal as shown in FIG. 1C are used. Among the apoplast-accumulation-type constructs, there are: an expression vector having the EGPh coding sequence (SEQ ID NO: 3) at the ‘EGs’ part of the vector in the diagram of FIG. 1B (ap-EGPh vector); an expression vector having the EGPf coding sequence with a modification in one of the SD-like sequences located second from the n-terminus (SEQ ID NO: 7), in the ‘EGs’ part (ap-SD2M vector); an expression vector having the EGPf coding sequence at the n-terminus thereof, and the coding sequence of the chitin binding domain of chitinase derived from Pyrococcus furiosus at the c-terminus thereof (SEQ ID NO: 9; ap-EGPfChiCBM vector).

Among the apoplast-accumulation-type constructs with an endoplasmic reticulum localization signal, there are: an expression vector having the EGPh coding sequence (SEQ ID NO: 3) at the ‘EGs’ part of the vector in the diagram of FIG. 1C (ap-EGPh-H vector); an expression vector having the EGPf coding sequence with a modification in one of the SD-like sequences located second from the n-terminus (SEQ ID NO: 7), in the ‘EGs’ part (ap-SD2M-H vector); an expression vector having the EGPf coding sequence at the n-terminus thereof, and the coding sequence of the chitin binding domain of chitinase derived from Pyrococcus furiosus at the c-terminus thereof (SEQ ID NO: 9; ap-EGPfChiCBM-H vector).

For the apoplastic-transfer signal peptide (ap) encoding sequence, the nucleotide sequence of SEQ ID NO: 11 was used, referring to the apoplastic-transfer signal peptide of Schaewen et al. (see Schaewen Av et al., 1990, The European Molecular Biology Organization Journal, 9, 3033-3044.)

For the endoplasmic reticulum localization signal peptide, the nucleotide sequence of SEQ ID NO: 12 was used.

First, an expression vector is introduced into agrobacterium (Agrobacterium tumefaciens) using freeze/thaw transformation method. Specifically, competent cells of agrobacterium EHA105 strain is thawed on ice, and one μg of the plasmid (expression vector) is added thereto, and mixed gently. The mixture is then flush-frozen using liquid nitrogen. Thereafter, the tube is thawed by incubating at 37° C. for 4 minutes, and 0.5 ml of SOC medium is added thereto. Thereafter, the mixture is incubated at 28° C. for 1 to 3 hours. The culture is then plated on LB-agar plates including 50 mg/L of kanamycin and 10 mg/L of phosphinotricin (PPT), and stationary cultured at 28° C. in an incubator for two days. Thereby transformant agrobacterium is obtained. The transformant agrobacterium is liquid cultured, and the contained plasmid is extracted and purified. The resulting plasmid is confirmed to be the expression vector originally used for the transformation, by PCR and restriction enzyme assays.

Next, transformant Arabidopsis is generated, using an Arabidopsis plant body cultivated for two month at 22° C. with 24 hours light period, and transformant agrobacterium grown in LB medium including 50 mg/L of kanamycin and 10 mg/L of PPT.

First, agrobacterium is collected from liquid culture at about OD₆₀₀=1, and resuspended into a buffer containing 5% sucrose and 0.05% Silwet solution. The Arabidopsis plant body is then soaked into the agrobacterium suspension, to facilitate infection to the seeds. After the maturation of the seeds, the seeds are harvested. Selection of transformant is performed using ½ MS medium including 50 mg/L of kanamycin and 10 mg/L of PPT, and thereby, transformant Arabidopsis is obtained. More specifically, 33 transformant plants with ap-EGPh vector (ap-EGPh1 to 33), 6 transformant plants with ap-SD2M vector (ap-SD2M1 to 6), 16 transformant plants with ap-EGPfChiCBM vector (ap-EGPfChiCBM1 to 16), 4 transformant plants with ap-EGPh-H vector (ap-EGPh-H1 to 4), 23 transformant plants with ap-SD2M-H vector (ap-SD2M-H1 to 23), and 4 transformant plants with ap-EGPfChiCBM-H vector (ap-EGPfChiCBM-H1 to 4), are obtained, respectively.

### Second Embodiment

### Extraction of Polypeptide Having Thermophilic Endoglucanase Activity from Transformant Arabidopsis

From the transformant Arabidopsis obtained in the first embodiment, polypeptide having thermophilic endoglucanase activity is extracted, and the thermophilic endoglucanase activity of the polypeptide was assayed.

The polypeptide extraction is performed by the method of Kawazu et al. and the method of Kimura et al. Specifically, 100 mg of transformant Arabidopsis leaves are milled under liquid nitrogen using mortar and pestle. Thereafter, one mL of cold extraction buffer (100 mM acetic acid, 10 mM EDTA, 0.1% TritonX-100, 0.1% Sarkosyl, 1 mM DTT, pH 5.6) is added thereto and thoroughly suspended. The mixture is then transferred to 2 ml microtubes and centrifuged at 15,000 rpm for 10 minutes at 4° C. The supernatant is recovered to yield the crude enzyme extract. The total protein concentration of the resulting crude enzyme extract was assayed using DC protein assay reagents (a product of Bio-Rad), which can measure samples including detergents. In the assay, BSA (bovine serum albumin) is used for the standard protein solution. As controls of the experiment, crude enzyme extracts are prepared using the same procedure from non-transformed wild-type Arabidopsis leaves.

The endoglucanase activity of the crude enzyme extracts are assayed by the DNSA method, using carboxyl methyl cellulose (CMC) as the substrate.

First, a reaction solution is prepared from sodium acetate buffer (pH 5.6) by adding a portion of crude enzyme extract containing 0.2 mg protein, and CMC to final concentration of 0.5%. Thereafter, the reaction solution is incubated at 85° C. for 16 hours. The amount of reducing sugar is quantified before and after the reaction. The thermophilic endoglucanase activity (unit/mg protein) of the crude enzyme extract is calculated from the amount of reducing sugar increased by the hydrolyzation by the crude enzyme extract. In the calculation, one unit is defined as the amount of enzyme required to produce one μg of glucose at 85° C. in one minute. The reducing sugar in the reaction solution is measured by using DNSA (3,5-dinitorosalicylic acid reagent). The standard curve for the quantitation is prepared using glucose.

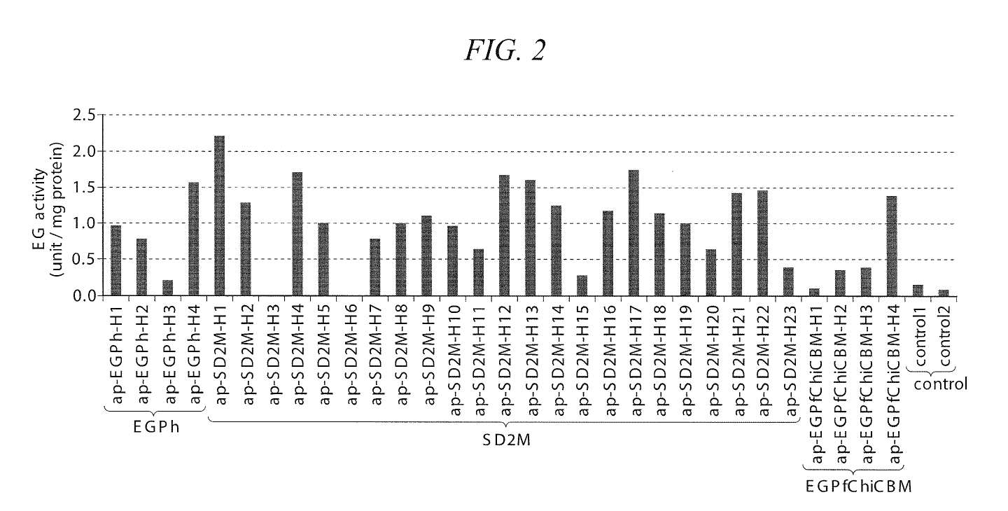

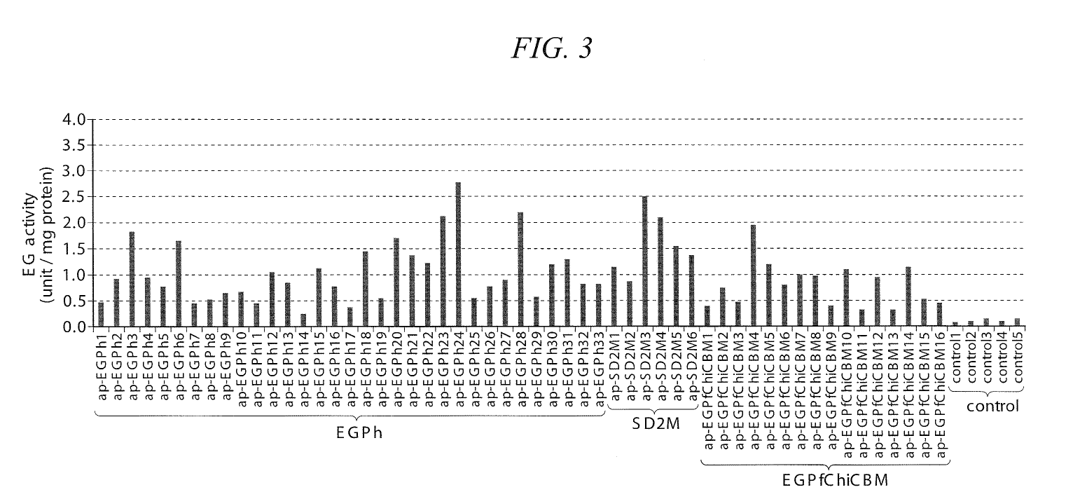

FIG. 2 shows thermophilic endoglucanase activities of the crude enzyme extracts derived from Arabidopsis transformants established using apoplast-accumulation-type constructs. FIG. 3 shows thermophilic endoglucanase activities of the crude enzyme extracts derived from Arabidopsis transformants established using apoplast-accumulation-type constructs with endoplasmic reticulum localization signal. The crude enzyme extracts derived from Arabidopsis transformants established using apoplast-accumulation-type constructs had considerably high thermophilic endoglucanase activity as compared to the control. On the other hand, among the Arabidopsis transformants established using apoplast-accumulation-type constructs with endoplasmic reticulum localization signal, although some plant individuals did not show significant thermophilic endoglucanase activity, many plant individuals showed considerably high thermophilic endoglucanase activity. In either constructs of apoplast-accumulation-type or apoplast-accumulation-type with endoplasmic reticulum localization signal, particular variation depending on the kind of polypeptide having thermophilic endoglucanase activity was not detected.

Therefore, from these results, it is clear that by performing transformation with expression vectors which include encoding sequence of a polypeptide having thermophilic endoglucanase activity, it is possible to obtain transformant plants expressing polypeptide having thermophilic endoglucanase activity at a high level.

### Third Embodiment

### Preparation of Transformant Rice

Transformant rice (Oryza sativa L.; cultivar name, Nihonbare) is prepared by the method of Nishimura using the transformant agrobacterium obtained in the first embodiment.

Specifically, after removing the outer shell from mature seeds, the seeds are treated with 70% ethanol for 30 seconds, and then with calcium hypochlorite solution at an effective chlorine concentration of 2% for 30 minutes to sterilize the surface thereof. The seeds are further treated with sterile distilled water for 5 to 7 times, and placed on N6D medium. The seed is cultivated at 30° C. under light (approximately 120 μmolm⁻² s⁻¹) for three to four weeks. The formed callus is transferred to a fresh N6D medium, and further incubated for three days.

Transformant agrobacterium solution is prepared, by incubating the transformant agrobacterium obtained in the first embodiment on AB medium including antibiotics for three days, and then suspending in AAM medium. The pre-cultured callus is soaked in the transformant agrobacterium solution for 90 seconds. After removing the excess agrobacterium solution by paper towels, the callus is placed on 2N6-AS medium. The callus is then co-incubated at 28° C. in darkness for two days. The resulting callus is washed with sterile distilled water for three to five times, transferred to N6D medium including 25 mg/L meropenem and 20 mg/L PPT, and incubated under light at 30° C. for four weeks, to obtain PPT resistant callus. The resulting ppt resistant callus is transferred to MS-NK medium including 25 mg/L meropenem and 20 mg/L PPT, and incubated for four weeks to obtain differentiated shoot cultures. The resulting shoot is transferred to MS-HF medium including 25 mg/L meropenem and 20 mg/L PPT and allowed to root, to obtain final transformant rice. The resulting transformant rice is transplanted into polypots and habituated in a growth chamber for two weeks. Thereafter, the transformant rice is transplanted into plastic pots and further cultivated in a closed greenhouse.

### Fourth Embodiment

### Preparation of Transformant Rice 2

In the fourth embodiment, the transformant agrobacterium is prepared essentially as described in the first embodiment, except for that, as the expression vector, instead of the ap-EGPh vector, another expression vector (cyt-EGPh vector) is used, in which EGPh encoding sequence (SEQ ID NO: 3) is inserted in the ‘EGs’ part in the FIG. 1A. Since the cyt-EGPh vector does not have apoplastic-transfer signal peptide at the n-terminus thereof, the expressed peptide is expected to be accumulated in the cytoplast.

Moreover, using the above explained transformant agrobacterium, transformant rice is obtained as in the third embodiment.

### Fifth Embodiment

### Extraction of Polypeptide Having Thermophilic Endoglucanase Activity from Transformant Rice

From the transformant rice obtained in the third and fourth embodiments, polypeptide having thermophilic endoglucanase activity is extracted, and the thermophilic endoglucanase activity of the polypeptide is assayed.

Specifically, the crude enzyme extract is prepared essentially as in the second embodiment, except for that instead of the transformant Arabidopsis leaves, transformant rice leaves are used. Thereafter, the endoglucanase activity of the crude enzyme extract is assayed by DNSA method, using carboxyl methyl cellulose (CMC) as substrate.

The following transformant rice are used: three transformant rice with the ap-EGPh vector obtained in the third embodiment (ap-EGPh1, 2, and 5); six transformant rice with the ap-SD2M vector (ap-SD2M1, 2, 4, 5, 8, 9); four transformant rice with the ap-EGPfChiCBM vector (ap-EGPfChiCBM1, 3 to 5); nine transformant rice with the ap-EGPh-H vector (ap-EGPh-H3, 4, 6 to 12); and three transformant rice with the cyt-EGPh vector obtained in the fourth embodiment (cyt-EGPh4, 6, 7). Moreover, for control samples, crude enzyme extracts are prepared in the same procedure using leaves of non-transformed wild type rice.

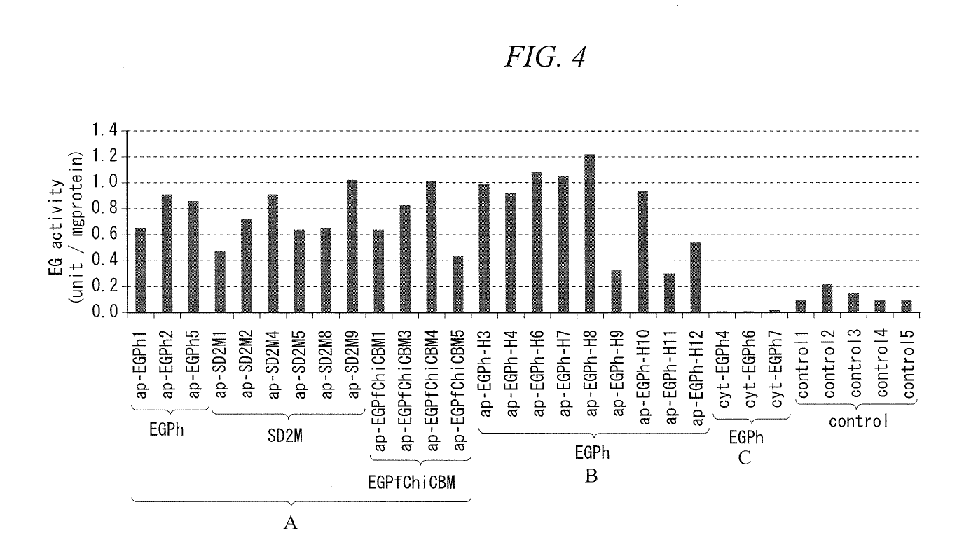

FIG. 4 shows thermophilic endoglucanase activities of the crude enzyme extracts derived from the obtained transformant rice. Group A represents the result using apoplast-accumulation-type constructs. Group B represents the result using apoplast-accumulation-type constructs with endoplasmic reticulum localization signal. Group C represents the result using cytoplast-accumulation-type constructs.

All of the crude enzyme extract obtained using the apoplast-accumulation-type constructs possess considerably high thermophilic endoglucanase activity as compared to the control samples. Moreover, the crude enzyme extract derived from the transformant rice obtained using apoplast-accumulation-type constructs with endoplasmic reticulum localization signal also possess considerably high thermophilic endoglucanase activity as compared to the control samples. On the other hand, significant thermophilic endoglucanase activity is not observed in the transformant rice obtained using cytoplast-accumulation-type construct. No particular variation depending on the kind of polypeptide having thermophilic endoglucanase activity was observed, for either the apoplast-accumulation-type constructs and the apoplast-accumulation-type constructs with endoplasmic reticulum localization signal.

Therefore, from these results, it is clear that by performing transformation with expression vectors which include encoding sequence of a polypeptide having thermophilic endoglucanase activity, it is possible to obtain transformant plants expressing polypeptide having thermophilic endoglucanase activity at a high level.

The transformant plant of the present invention can be utilized in the field of biomass ethanol production, since it can simultaneously provide both cellulose and thermophilic endoglucanase suitable for cellulose hydrolyzation.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A transformant plant transformed with an expression vector, the expression vector including a nucleotide sequence encoding a first polypeptide which has thermophilic endo-1,4-beta-glucanase activity, so that the polypeptide is capable of being expressed in a host cell of the transformant plant.

2. The transformant plant according to claim 1, wherein the first polypeptide further includes an amino acid sequence of a chitin binding domain of a chitinase.

3. The transformant plant according to claim 1, wherein the first polypeptide is a polypeptide having an amino acid sequence selected from the group consisting of:
(a) an amino acid sequence set forth in SEQ ID NO: 2;
(b) an amino acid sequence set forth in SEQ ID NO: 2 including substitution, deletion, insertion, and/or addition of one or several of amino acids in the amino acid sequence.

4. The transformant plant according to claim 1, wherein the first polypeptide is a polypeptide having an amino acid sequence selected from the group consisting of:
(a) an amino acid sequence set forth in SEQ ID NO: 4;
(b) an amino acid sequence set forth in SEQ ID NO: 4 including substitution, deletion, insertion, and/or addition of one or several of amino acids in the amino acid sequence.

5. The transformant plant according to claim 1, wherein the first polypeptide is a polypeptide having an amino acid sequence selected from the group consisting of:
(a) an amino acid sequence set forth in SEQ ID NO: 6;
(b) an amino acid sequence set forth in SEQ ID NO: 6 including substitution, deletion, insertion, and/or addition of one or several of amino acids in the amino acid sequence.

6. The transformant plant according to claim 1, wherein the nucleotide sequence is selected from the group consisting of:
(a) a nucleotide sequence set forth in SEQ ID NO: 7;
(b) a nucleotide sequence set forth in SEQ ID NO: 7 including substitution, deletion, insertion, and/or addition of one or several of nucleotide in the nucleotide sequence.

7. The transformant plant according to claim 1, wherein the first polypeptide is a polypeptide having an amino acid sequence selected from the group consisting of:
(a) an amino acid sequence set forth in SEQ ID NO: 10;
(b) an amino acid sequence set forth in SEQ ID NO: 10 including substitution, deletion, insertion, and/or addition of one or several of amino acids in the amino acid sequence.

8. The transformant plant according to claim 1, wherein the first polypeptide further includes an apoplastic-transfer signal peptide at the amino-terminus thereof.

9. The transformant plant according to claim 1, wherein the first polypeptide further includes an endoplasmic reticulum localization signal peptide at the carboxyl-terminus thereof.

10. The transformant plant according to claim 1, wherein the plant belongs to family Brassicaceae.

11. The transformant plant according to claim 10, wherein the plant is Arabidopsis thaliana.

12. The transformant plant according to claim 1, wherein the plant belongs to family Poaceae.

13. The transformant plant according to claim 12, wherein the plant is rice.


SEQUENCE LISTING (15 sequences)

SEQ ID NO: 1 - DNA, 1377 bp, Pyrococcus horikoshii
  CDS (1)..(1374)
atggaggggaatactattcttaaaatcgtactaatttgcactattttagcaggcctattcgggcaagtcgtgccagtatatgcagaaaatacaacatatcaaacaccgactggaatttactacgaagtgagaggagatacgatatacatgattaatgtcaccagtggagaggaaactcccattcatctctttggtgtaaactggtttggctttgaaacacctaatcatgtagtgcacggactttggaagagaaactgggaagacatgcttcttcagatcaaaagcttaggcttcaatgcaataagacttcctttctgtactgagtctgtaaaaccaggaacacaaccaattggaatagattacagtaaaaatccagatcttcgtggactagatagcctacagattatggaaaagatcataaagaaggccggagatcttggtatctttgtcttactcgactatcataggataggatgcactcacatagaacccctctggtacacggaagacttctcagaggaagactttattaacacatggatagaggttgccaaaaggttcggtaagtactggaacgtaataggggctgatctaaagaatgagcctcatagtgttacctcacccccagctgcttatacagatggtaccggggctacatggggtatgggaaaccctgcaaccgattggaacttggcggctgagaggataggaaaagcgattctgaaggttgcccctcattggttgatattcgtggaggggacacaatttactaatccgaagactgacagtagttacaaatggggctacaacgcttggtggggaggaaatctaatggccgtaaaggattatccagttaacttacctaggaataagctagtatacagccctcacgtatatgggccagatgtctataatcaaccgtactttggtcccgctaagggttttccggataatcttccagatatctggtatcaccactttggatacgtaaaattagaactaggatattcagttgtaataggagagtttggaggaaaatatgggcatggaggcgatccaagggatgttatatggcaaaataagctagttgattggatgatagagaataaattttgtgatttcttttactggagctggaatccagatagtggagataccggagggattctacaggatgattggacaacaatatgggaagataagtataataacctgaagagattgatggatagttgttccaaaagttcttcaagtactcaatccgttattcggagtaccacccctacaaagtcaaatacaagtaagaagatttgtggaccagcaattcttatcatcctagcagtattctctcttctcttaagaagggctcccaggtag

SEQ ID NO: 2 - protein, 458 aa, Pyrococcus horikoshii
MEGNTILKIVLICTILAGLFGQVVPVYAENTTYQTPTGIYYEVRGDTIYMINVTSGEETPIHLFGVNWFGFETPNHVVHGLWKRNWEDMLLQIKSLGFNAIRLPFCTESVKPGTQPIGIDYSKNPDLRGLDSLQIMEKIIKKAGDLGIFVLLDYHRIGCTHIEPLWYTEDFSEEDFINTWIEVAKRFGKYWNVIGADLKNEPHSVTSPPAAYTDGTGATWGMGNPATDWNLAAERIGKAILKVAPHWLIFVEGTQFTNPKTDSSYKWGYNAWWGGNLMAVKDYPVNLPRNKLVYSPHVYGPDVYNQPYFGPAKGFPDNLPDIWYHHFGYVKLELGYSVVIGEFGGKYGHGGDPRDVIWQNKLVDWMIENKFCDFFYWSWNPDSGDTGGILQDDWTTIWEDKYNNLKRLMDSCSKSSSSTQSVIRSTTPTKSNTSKKICGPAILIILAVFSLLLRRAPR

SEQ ID NO: 3 - DNA, 1296 bp, Artificial Sequence
  Description of Artificial Sequence Synthetic polynucleotide
atggaaaatacaacatatcaaacaccgactggaatttactacgaagtgagaggagatacgatatacatgattaatgtcaccagtggagaggaaactcccattcatctctttggtgtaaactggtttggctttgaaacacctaatcatgtagtgcacggactttggaagagaaactgggaagacatgcttcttcagatcaaaagcttaggcttcaatgcaataagacttcctttctgtactgagtctgtaaaaccaggaacacaaccaattggaatagattacagtaaaaatccagatcttcgtggactagatagcctacagattatggaaaagatcataaagaaggccggagatcttggtatctttgtcttactcgactatcataggataggatgcactcacatagaacccctctggtacacggaagacttctcagaggaagactttattaacacatggatagaggttgccaaaaggttcggtaagtactggaacgtaataggggctgatctaaagaatgagcctcatagtgttacctcacccccagctgcttatacagatggtaccggggctacatggggtatgggaaaccctgcaaccgattggaacttggcggctgagaggataggaaaagcgattctgaaggttgcccctcattggttgatattcgtggaggggacacaatttactaatccgaagactgacagtagttacaaatggggctacaacgcttggtggggaggaaatctaatggccgtaaaggattatccagttaacttacctaggaataagctagtatacagccctcacgtatatgggccagatgtctataatcaaccgtactttggtcccgctaagggttttccggataatcttccagatatctggtatcaccactttggatacgtaaaattagaactaggatattcagttgtaataggagagtttggaggaaaatatgggcatggaggcgatccaagggatgttatatggcaaaataagctagttgattggatgatagagaataaattttgtgatttcttttactggagctggaatccagatagtggagataccggagggattctacaggatgattggacaacaatatgggaagataagtataataacctgaagagattgatggatagttgttccaaaagttcttcaagtactcaatccgttattcggagtaccacccctacaaagtcaaatacaagtaagaagatttgtggaccagcaattcttatcatcctagcagtattctctcttctcttaagaagggctcccaggtag

SEQ ID NO: 4 - protein, 431 aa, Artificial Sequence
  Description of Artificial Sequence Synthetic polypeptide
MENTTYQTPTGIYYEVRGDTIYMINVTSGEETPIHLFGVNWFGFETPNHVVHGLWKRNWEDMLLQIKSLGFNAIRLPFCTESVKPGTQPIGIDYSKNPDLRGLDSLQIMEKIIKKAGDLGIFVLLDYHRIGCTHIEPLWYTEDFSEEDFINTWIEVAKRFGKYWNVIGADLKNEPHSVTSPPAAYTDGTGATWGMGNPATDWNLAAERIGKAILKVAPHWLIFVEGTQFTNPKTDSSYKWGYNAWWGGNLMAVKDYPVNLPRNKLVYSPHVYGPDVYNQPYFGPAKGFPDNLPDIWYHHFGYVKLELGYSVVIGEFGGKYGHGGDPRDVIWQNKLVDWMIENKFCDFFYWSWNPDSGDTGGILQDDWTTIWEDKYNNLKRLMDSCSKSSSSTQSVIRSTTPTKSNTSKKICGPAILIILAVFSLLLRRAPR

SEQ ID NO: 5 - DNA, 1170 bp, Artificial Sequence
  Description of Artificial Sequence Synthetic polynucleotide
atggaaaatacaacatatcaaacaccgactggaatttactacgaagtgagaggagatacgatatacatgattaatgtcaccagtggagaggaaactcccattcatctctttggtgtaaactggtttggctttgaaacacctaatcatgtagtgcacggactttggaagagaaactgggaagacatgcttcttcagatcaaaagcttaggcttcaatgcaataagacttcctttctgtactgagtctgtaaaaccaggaacacaaccaattggaatagattacagtaaaaatccagatcttcgtggactagatagcctacagattatggaaaagatcataaagaaggccggagatcttggtatctttgtcttactcgactatcataggataggatgcactcacatagaacccctctggtacacggaagacttctcagaggaagactttattaacacatggatagaggttgccaaaaggttcggtaagtactggaacgtaataggggctgatctaaagaatgagcctcatagtgttacctcacccccagctgcttatacagatggtaccggggctacatggggtatgggaaaccctgcaaccgattggaacttggcggctgagaggataggaaaagcgattctgaaggttgcccctcattggttgatattcgtggaggggacacaatttactaatccgaagactgacagtagttacaaatggggctacaacgcttggtggggaggaaatctaatggccgtaaaggattatccagttaacttacctaggaataagctagtatacagccctcacgtatatgggccagatgtctataatcaaccgtactttggtcccgctaagggttttccggataatcttccagatatctggtatcaccactttggatacgtaaaattagaactaggatattcagttgtaataggagagtttggaggaaaatatgggcatggaggcgatccaagggatgttatatggcaaaataagctagttgattggatgatagagaataaattttgtgatttcttttactggagctggaatccagatagtggagataccggagggattctacaggatgattggacaacaatatgggaagataagtataataacctgaagagattgatggatagttgttccaaaagttcttag

SEQ ID NO: 6 - protein, 389 aa, Artificial Sequence
  Description of Artificial Sequence Synthetic polypeptide
MENTTYQTPTGIYYEVRGDTIYMINVTSGEETPIHLFGVNWFGFETPNHVVHGLWKRNWEDMLLQIKSLGFNAIRLPFCTESVKPGTQPIGIDYSKNPDLRGLDSLQIMEKIIKKAGDLGIFVLLDYHRIGCTHIEPLWYTEDFSEEDFINTWIEVAKRFGKYWNVIGADLKNEPHSVTSPPAAYTDGTGATWGMGNPATDWNLAAERIGKAILKVAPHWLIFVEGTQFTNPKTDSSYKWGYNAWWGGNLMAVKDYPVNLPRNKLVYSPHVYGPDVYNQPYFGPAKGFPDNLPDIWYHHFGYVKLELGYSVVIGEFGGKYGHGGDPRDVIWQNKLVDWMIENKFCDFFYWSWNPDSGDTGGILQDDWTTIWEDKYNNLKRLMDSCSKSS

SEQ ID NO: 7 - DNA, 1170 bp, Artificial Sequence
  Description of Artificial Sequence Synthetic polynucleotide
atggaaaatacaacatatcaaacaccgactggaatttactacgaagtgagaggagatacgatatacatgattaatgtcaccagtggagaggaaactcccattcatctctttggtgtaaactggtttggctttgaaacacctaatcatgtagtgcacggactttggaagagaaactgggaagacatgcttcttcagatcaaaagcttaggcttcaatgcaataagacttcctttctgtactgagtctgtaaaaccaggaacacaaccaattggaatagattacagtaaaaatccagatcttcgtggactagatagcctacagattatggaaaagatcataaagaaggccggagatcttggtatctttgtcttactcgactatcataggataggatgcactcacatagaacccctctggtacacggaagacttctcagaggaagactttattaacacatggatagaggttgccaaaaggttcggtaagtactggaacgtaataggggctgatctaaagaatgagcctcatagtgttacctcacccccagctgcttatacagatggtaccggggctacatggggtatgggaaaccctgcaaccgattggaacttggcggctgagaggataggaaaagcgattctgaaggttgcccctcattggttgatattcgtggaggggacacaatttactaatccgaagactgacagtagttacaaatggggctacaacgcttggtggggtggtaatctaatggccgtaaaggattatccagttaacttacctaggaataagctagtatacagccctcacgtatatgggccagatgtctataatcaaccgtactttggtcccgctaagggttttccggataatcttccagatatctggtatcaccactttggatacgtaaaattagaactaggatattcagttgtaataggagagtttggaggaaaatatgggcatggaggcgatccaagggatgttatatggcaaaataagctagttgattggatgatagagaataaattttgtgatttcttttactggagctggaatccagatagtggagataccggagggattctacaggatgattggacaacaatatgggaagataagtataataacctgaagagattgatggatagttgttccaaaagttcttag

SEQ ID NO: 8 - protein, 1075 aa, Pyrococcus furiosus
MKTRMLGIVLAWLVVLSLVSPTISLFYPVSAQQTVQLDGYAISWDVVNLTWTPVENVNGYEIYRSTSMENLVSLQNLLVYVNWSSYPKYEPGKEYNQGDIVEYNGKLYKAKYWTTSPPSDDPYGSWEYLGEAEPTTNYLDQFRLKPETTYYYAVVPVFKDGSRGEPSNIIRITTPKEPFRVVVYYISWGIYARKFFPEDIPFEKVTHVNYAFLNPKEDGTVDFYDTWADPQNLEKFKELKKKYPQVKILISVGGWTLSKYFSVIAADPAKRERFARTALEIIRKYNLDGLDIDWEYPGGGGMEGNYVSPDDGKNFVLLVKTVREIFDQAELEDKKEYLLTAAVPADPVKAARINWTEAMKYLDFINVMTYDYHGAWDPITGHLAPLYADPNAPYEDPNIKWNFNVNASIQWYLKHGVNPKQLGLGLPFYGRSFANVPPENNGLYQPFSGTPAGTWGPAYETHGVMDYWDIEEKRASGQYNYYWDPVAMVPWLYSPSLKIFISYDDKKSIGIKVDYALKYNLGGVMVWEITADRKPGTNSHPLLDTILEHIAQGGGVVPTPSPTPTPTPTPTPTPTTTTTTSTPTPTTTTTPSPTTTTTTTTTTTTTSTTTTPTTTTTPVPVSGSLEVKVNDWGSGAEYDVTLNLDGQYDWTVKVKLAPGATVGSFWSANKQEGNGYVIFTPVSWNKGPTATFGFIVNGPQGDKVEEITLEINGQVIDIWTPTGGTTPTPTTTTTSTPTPSQTPTPTPTPTPTPTPTPTLTPTPLPGNANPIPEHFFAPYIDMSLSVHKPLVEYAKLTGTKYFTLAFILYSSVYNGPAWAGSIPLEKFVDEVRELREIGGEVIIAFGGAVGPYLCQQASTPEQLAEWYIKVIDTYNATYLDFDIEAGIDADKLADALLIVQRERPWVKFSFTLPSDPGIGLAGGYGIIETMAKKGVRVDRVNPMTMDYYWTPSNAENAIKVAENVFRQLKQIYPEKSDEEIWKMIGLTPMIGVNDDKSVFTLEDAQQLVDWAIQHKIGSLAFWSVDRDHPGPTGEVSPLHRGTNDPDWAFSHVFVKFMEAFGYTFSAQTSEASVPT

SEQ ID NO: 9 - DNA, 1503 bp, Artificial Sequence
  Description of Artificial Sequence Synthetic polynucleotide
atggaaaatacaacatatcaaacaccgactggaatttactacgaagtgagaggagatacgatatacatgattaatgtcaccagtggagaggaaactcccattcatctctttggtgtaaactggtttggctttgaaacacctaatcatgtagtgcacggactttggaagagaaactgggaagacatgcttcttcagatcaaaagcttaggcttcaatgcaataagacttcctttctgtactgagtctgtaaaaccaggaacacaaccaattggaatagattacagtaaaaatccagatcttcgtggactagatagcctacagattatggaaaagatcataaagaaggccggagatcttggtatctttgtcttactcgactatcataggataggatgcactcacatagaacccctctggtacacggaagacttctcagaggaagactttattaacacatggatagaggttgccaaaaggttcggtaagtactggaacgtaataggggctgatctaaagaatgagcctcatagtgttacctcacccccagctgcttatacagatggtaccggggctacatggggtatgggaaaccctgcaaccgattggaacttggcggctgagaggataggaaaagcgattctgaaggttgcccctcattggttgatattcgtggaggggacacaatttactaatccgaagactgacagtagttacaaatggggctacaacgcttggtggggaggaaatctaatggccgtaaaggattatccagttaacttacctaggaataagctagtatacagccctcacgtatatgggccagatgtctataatcaaccgtactttggtcccgctaagggttttccggataatcttccagatatctggtatcaccactttggatacgtaaaattagaactaggatattcagttgtaataggagagtttggaggaaaatatgggcatggaggcgatccaagggatgttatatggcaaaataagctagttgattggatgatagagaataaattttgtgatttcttttactggagctggaatccagatagtggagataccggagggattctacaggatgattggacaacaatatgggaagataagtataataacctgaagagattgatggatagttgttccaaaagttcttcaggatccaccactacaactacccctgtcccagtctcaggatctctagaggtaaaggtaaacgattggggtagtggtgctgagtatgatgtgactcttaatttggatggacagtatgactggactgtgaaggttaaactagccccaggagccactgtaggaagcttctggagcgctaacaaacaagaggggaatggctatgtcatcttcactccagtaagctggaataaagggccgacagcaacatttggattcatagtaaacggaccacaaggagacaaagtagaagaaataaccctagaaataaacggacaagtaattgacatatggacatga

SEQ ID NO: 10 - protein, 500 aa, Artificial Sequence
  Description of Artificial Sequence Synthetic polypeptide
MENTTYQTPTGIYYEVRGDTIYMINVTSGEETPIHLFGVNWFGFETPNHVVHGLWKRNWEDMLLQIKSLGFNAIRLPFCTESVKPGTQPIGIDYSKNPDLRGLDSLQIMEKIIKKAGDLGIFVLLDYHRIGCTHIEPLWYTEDFSEEDFINTWIEVAKRFGKYWNVIGADLKNEPHSVTSPPAAYTDGTGATWGMGNPATDWNLAAERIGKAILKVAPHWLIFVEGTQFTNPKTDSSYKWGYNAWWGGNLMAVKDYPVNLPRNKLVYSPHVYGPDVYNQPYFGPAKGFPDNLPDIWYHHFGYVKLELGYSVVIGEFGGKYGHGGDPRDVIWQNKLVDWMIENKFCDFFYWSWNPDSGDTGGILQDDWTTIWEDKYNNLKRLMDSCSKSSSGSTTTTTPVPVSGSLEVKVNDWGSGAEYDVTLNLDGQYDWTVKVKLAPGATVGSFWSANKQEGNGYVIFTPVSWNKGPTATFGFIVNGPQGDKVEEITLEINGQVIDIWT

SEQ ID NO: 11 - DNA, 230 bp, Artificial Sequence
  Description of Artificial Sequence Synthetic polynucleotide
aattatccatcatggatgttcacaaggaagttaatttcgttgcttacctactaattgttcttggtaagattttcctttactcctttttttttttttttaaaaaaaattcttggtttatacatatatatatatatacacaagtagttttatatttttcctttatattatatttgtttgtaggattattggtacttgtaagcgcgatggagcatgttgatgcgaaggcttgc

SEQ ID NO: 12 - DNA, 12 bp, Artificial Sequence
  Description of Artificial Sequence Synthetic oligonucleotide
catgatgagctt

SEQ ID NO: 13 - protein, 389 aa, Artificial Sequence; identical to SEQ ID NO: 6
  Description of Artificial Sequence Synthetic polypeptide

SEQ ID NO: 14 - protein, 34 aa, Artificial Sequence
  Description of Artificial Sequence Synthetic peptide
MDVHKEVNFVAYLLIVLGLLVLVSAMEHVDAKAC

SEQ ID NO: 15 - protein, 4 aa, Artificial Sequence
  Description of Artificial Sequence Synthetic peptide
HDEL